(19) United States
(12) Patent Application Publication
(10) Pub. No.: US 2006/0068019 A1
(43) Pub. Date: Mar. 30, 2006

(54) Coated polyunsaturated fatty acid-containing particles and coated liquid pharmaceutical-containing particles

(76) Inventors: Sean Mark Dalziel, Wilmington, DE (US); Thomas E Friedmann, Hockessin, DE (US); George A Schurr, Newark, DE (US)

Correspondence Address: E I Du Pont De Nemours and Company, Legal Patents, Wilmington, DE, 19805, US

(21) Appl. No.: 10/523,225

(22) PCT Filed: Aug. 14, 2003
(86) PCT No.: PCT/US03/25873

§ 371 Date: Jan. 31, 2005

Related U.S. Application Data
(60) Provisional application No. 60/403,598, filed on Aug. 14, 2002.

(51) Int. Cl. A61K 9/50; A23F 5/00; A61K 9/16

(52) U.S. Cl. 424/490; 426/302

(57) ABSTRACT

A process for coating a polyunsaturated fatty acid (PUFA)-containing carrier particle or a PUFA matrix particle, or a liquid pharmaceutical-containing carrier particle or a liquid pharmaceutical matrix particle. Also disclosed are such particles made by the process of the invention and foods, pharmaceuticals, beverages, nutritional supplements, infant formula, pet food and animal feed with incorporate such particles.

This application claims the benefit of U.S. Provisional Application No. 60/403598, filed Aug. 14, 2002.

### FIELD OF THE INVENTION

This invention is in the field of particle coating, specifically, coating of polyunsaturated fatty acid containing particles and coating of liquid pharmaceutical-containing particles. The coated products are useful in the pharmaceutical, nutriceutical and food industries.

### BACKGROUND OF THE INVENTION

There is a need in the art for a means of economically handling, preserving, delivering and measuring liquids, both aqueous and nonaqueous liquids such as food oils and pharmaceuticals, in many commercial applications including in the food and pharmaceutical industries. One such currently employed method involves the coating of liquids onto small particles, which particles, optimally, retain the stability and handling characteristics of solid flowable powders. Further, liquids processed into solid powder form have been found to be less susceptible to deterioration through excessive temperature, volatilization or reaction with oxygen. The particles that are coated may be carrier particles or active particles. Active particles generally are those that will be part of the desired material to be delivered. Carrier particles generally are those that are relatively inert in the sense that they are not part of the desired material to be delivered.

One example of an “active” particle is one that is comprised of a liquid pharmaceutically-active material. A further example of an active particle is one that is comprised of polyunsaturated fatty acids (PUFA). The human body is capable of producing most of the fatty acids which it requires to function. Two polyunsaturated fatty acids, eicosapentaenoic acid (EPA) and docosahexaenoic acid (DHA), however, cannot be synthesized efficiently by the human body and, thus, have to be supplied through the diet. Since the human body cannot produce adequate quantities of these polyunsaturated fatty acids, they are called essential fatty acids.

Two main families of PUFAs are the omega-3 fatty acids and the omega-6 fatty acids. EPA and DHA are very important omega-3 fatty acids. Fish oil is known to be one of the best sources of these omega-3 fatty acids. PUFAs are important components of the plasma membrane of the cell, where they may be found in such forms as phospholipids. PUFAs are necessary for proper development, particularly in the developing infant brain, and for tissue formation and repair. PUFAs also serve as precursors to other molecules of importance in human beings and animals, including the prostacyclins, eicosanoids, leukotrienes and prostaglandin.

Four major long chain PUFAs of importance include DHA and EPA which are primarily found in different types of fish oil, gamma-linolenic acid (GLA) which is found in the seeds of a number of plants, including evening primrose (Oenothera biennis), borage (Borago officinalis) and black currants (Ribes nigrum), and stearidonic acid (SDA), which is found in marine oils and plants seeds. Both GLA and another important long chain PUFA, arachidonic acid (ARA), are found in filamentous fungi. ARA can be purified from animal tissues including liver and adrenal gland. GLA, ARA, EPA and SDA are themselves, or are dietary precursors to, important long chain fatty acids involved in prostaglandin synthesis, in treatment of heart disease, and in development of brain tissue.

Research has shown that omega-3 fatty acids reduce the risk of heart disease as well as having a positive effect on children's development. Results have been disclosed indicating the positive effect of these fatty acids on certain mental illnesses, autoimmune diseases and joint complaints. Thus, there are many health benefits associated with a diet supplemented with these fatty acids.

Unfortunately, PUFAs are very susceptible to oxidation due to the high degree of unsaturation. When oxidized, the fatty acids turn rancid producing an unpleasant smell and taste. This means that for PUFAs to be incorporated into food components, they have to be protected against oxidation. The coating of these materials affords protection to these ingredients and allows them to be delivered at the target site at the required time. Shelf-life and stability of PUFAs is improved due to the inhibition of oxidation. Other benefits of coating include the ease of handling of the material(s) due to the small particulate, powder form of the coated PUFA-containing material or coated PUFA matrix particle and the suitability for incorporation at any of a variety of stages of preparation of many differing types of foodstuffs and nutritional compositions.

Microencapsulation has been defined as a process by which small particles (generally between 1 to 1000 microns in diameter) of solid, liquid or gas are packaged within a secondary material to form a microcapsule. (Sanguansri et al., Microencapsulation for Innovative Ingredients A Scoping Study: Opportunities for Research into the Microencapsulation of Food Ingredients, Food Science Australia, May 2001).

Vasishtha, Prepared Foods, “Microencapsulation: Delivering a market advantage (July 2002) provides an overview of the types of core and coating materials and encapsulation techniques used in the industry. A variety of encapsulation processes are mentioned and include both physical and chemical techniques. Examples of physical techniques mentioned are spray-drying, the spinning disc and coextrusion processes. Examples of chemical techniques mentioned are phase separation, gelation and coacervation.

U.S. Pat. No. 6,048,557, issued to Van Den Berg et al. on Apr. 11, 2002, describes PUFA encapsulated solid carrier particle for use as a foodstuff. Specifically, solid carrier particles are provided on to which have been encapsulated, or absorbed, at least one PUFA in a liquid form.

WO 01174175, published Oct. 11, 2001, described encapsulation of food ingredients, in particular, oxygen sensitive oils or oil soluble ingredients.

U.S. Pat. No. 4,895,725, issued to Kantor et al. on Jan. 23, 1990, describes microencapsulation of fish oils which are formed by preparing an emulsion of an oil-based biologically active compound and a non-oil soluble enteric coating in a basic solution, atomizing the emulsion into an acidic aqueous solution, and separating the precipitated microcapsules from the acidic aqueous solution.

U.S. Pat. No. 6,234,464 B1, issued to Krumbholz et al. on May 22, 2001, describes microencapsulated unsaturated fatty acid or fatty acid compound or mixture thereof involving two layers. The inner layer is composed of gelatin A, gelatin B, casein or an alginate or of a derivative or salt of one of these polymers. The outer layer is composed of gelatin B, gum arabic, pectin or chitosan or a derivative or salt of one of these polymers.

U.S. Pat. No. 4,217,370, issued to Rawlings et al. on Aug. 12, 1980, describes lipid-containing feed supplements and foodstuffs made by admixing a lipid material to form an emulsion and adjusting the pH to lower it to its isoelectric point, thereby aggregating the protein and simultaneously microencapsulating the lipid.

U.S. Pat. No. 5,087,461, issued to Levine et al. on Feb. 11, 1992, describes a spray-dried composition comprising at least one volatile component in a carrier and in the form of an amorphous powder, is further encapsulated in a glassy matrix formed from a mixture of four different carbohydrates.

An apparatus and process for coating small solid particles, such as powdery or granular materials, are described in WO 97/07879 published Mar. 6, 1997 and assigned to E. I. du Pont de Nemours and Company. This process involves metering a liquid composition comprising a coating material, where the liquid composition is either a solution, slurry or melt, into a flow restrictor and injecting a gas stream through the flow restrictor concurrently with the metering of the liquid composition to create a zone of turbulence at the outlet of the flow restrictor, thereby atomizing the liquid composition. The gas stream is heated prior to injecting it through the flow restrictor. A solid particle is added to the zone of turbulence concurrently with the metering of the liquid composition and the injection of the heated gas to mix the solid particle with the atomized liquid composition. The mixing at the zone of turbulence coats the solid particle with the coating material.

WO 97/07676 to E. I. du Pont de Nemours and Company discloses the apparatus of WO 97/07879, along with the use of the apparatus in a process for coating crop protection solid particles. Coatings are water-insoluble, and coating thicknesses are represented by weight percent rather than thickness.

U.S. Pat. No. 6,015,773, issued to Wysong et al. on Jan. 18, 2000, describes a crop protection composition comprising a mononucleate crop protection solid particle coated with water-insoluble coating material having a diameter in the range from 0.5 to 50 micrometers. This composition is made by a process which results in substantial non-agglomeration of the coated particles.

Applicants' assignee's copending application having application Ser. No. 10/174687, filed Jun. 19, 2002 and having Attorney Docket Number CL-1879 US NA discloses a process for dry coating a food particle having its largest diameter in the range from 0.5 mm to 20.0 mm with a liquid coating material. The coated food particle has a moisture level that is substantially the same as the moisture level of the uncoated food particle. Also disclosed is a process for coating a frozen liquid particle having a size in the range from 5 micrometers to 5 millimeters with a liquid coating material.

Applicants' assignees' copending, concurrently filed herewith provisional applications having Attorney Docket numbers CL2101, CL2149, CL2150, CL2178 and PTI sp1255 disclose subject matter related to the present application, and are specifically incorporated herein by reference.

U.S. Pat. No. 6,224,939 B1 issued to Cherukuri et al May 1, 2002 describes a method an apparatus for the coating of feedstock, wherein a solid matrix additive is spray injected in a free-flow condition.

U.S. Pat. Nos. 3,241,520 and 3,253,944 disclose a particle coating method wherein relatively large pellets, granules and particles are suspended in a stream of air while coating material in a liquid form is mixed with the particles.

Shahidi et al., Critical Reviews in Food Science and Nutrition, 33(6): 501-547 (1993) presents a review of the art of microcoating of food ingredients.

U.S. Pat. No. 4,848,673, issued to Masuda et al. on Jul. 18, 1989, describes a fluidized granulating and coating apparatus and method are recirculated through the treating vessel to ensure high stability of the granulating and coating operation.

### SUMMARY OF THE INVENTION

The present invention includes a process for coating a polyunsaturated fatty acid (PUFA)-containing carrier particle or a PUFA matrix particle, the process comprising the steps of:
(a) metering a liquid coating material through a flow restrictor;
(b) injecting a gas stream through the flow restrictor concurrently with step (a) to (i) atomize the liquid coating material and (ii) create turbulent flow of the gas stream and the atomized liquid coating material, wherein the gas stream is optionally heated; and
(c) adding a PUFA-containing carrier particle or a PUFA matrix particle to the turbulent flow region concurrently with steps (a) and (b), wherein the PUFA-containing carrier particle or the PUFA matrix particle mixes with the atomized liquid coating material to provide an coated PUFA-containing carrier particle or a PUFA matrix particle.

The present invention further includes a process for coating a liquid pharmaceutical-containing carrier particle or a liquid pharmaceutical matrix particle, the process comprising the steps of:
(a) metering a liquid coating material through a flow restrictor;
(b) injecting a gas stream through the flow restrictor concurrently with step (a) to (i) atomize the liquid coating material and (ii) create turbulent flow of the gas stream and the atomized liquid coating material, wherein the gas stream is optionally heated; and
(c) adding a liquid pharmaceutical-containing carrier particle or a liquid pharmaceutical matrix particle to the turbulent flow region concurrently with steps (a) and (b), wherein the liquid pharmaceutical-containing carrier particle or the liquid pharmaceutical matrix particle mixes with the atomized liquid coating material to provide an coated pharmaceutical-containing carrier particle or a pharmaceutical matrix particle.

This invention can be practiced with carrier particles selected from the nonexhaustive group consisting of a protein, fumed silica, titanium dioxide, calcium carbonate, a carbohydrate, a food particle, minerals, salts, lipids, antioxidants, a solid pharmaceutical particle, or any solid particle capable of being loaded with a PUFA or a liquid pharmaceutical.

As used by Applicants for purposes of this disclosure, pharmaceuticals can be considered to include, but are not limited to, nutraceuticals, vitamins, supplements, minerals, enzymes, probiotics, bronchodilators, anabolic steroids, analeptics, analgesics, proteins, peptides, antibodies, vaccines, anesthetics, antacids, antihelmintics, anti-arrthymics, antibiotics, anticoagulants, anticolonergics, anticonvulsants, antidepressants, antidiabetics, antidiarrheals, anti-emetics, anti-epileptics, antihistamines, antihormones, antihypertensives, anti-inflammatories, antimuscarinics, antimycotics, antineoplastics, anti-obesity drugs, antiprotozoals, antipsychotics, antispasmotics, anti-thrombics, antithyroid drugs, antitussives, antivirals, anxiolytics, astringents, beta-adrenergic receptor blocking drugs, bile acids, bronchospasmolytic drugs, calcium channel blockers, cardiac glycosides, contraceptives, corticosteriods, diagnostics, digestives, diuretics, dopaminergics, electrolytes, emetics, haemostatic drugs, hormones, hormone replacement therapy drugs, hypnotics, hypoglycemic drugs, immunosuppressants, impotence drugs, laxatives, lipid regulators, muscle relaxants, pain relievers, parasympathicolytics, parasympathicomimetics, prostagladins, psychostimulants, sedatives, sex steroids, spasmolytics, sulfonamides, sympathicolytics, sympathicomimetics, sympathomimetics, thyreomimetics, thyreostatic drugs, vasodialators, and xanthines. Many such pharmaceuticals can be formulated into solid carrier particles or solid matrix particles, suitable for being loaded with a liquid pharmaceutical or PUFA, and then subsequently coated by the process of the invention. Regarding the process of loading liquid pharmaceutical materials onto solid carrier particles or porpous matrix particles, Applicants specifically incorporate by reference herein the concurrently-filed and co-owned application having attorney docket number CL2178 and entitled :Process for Coating a Pharmaceutical Particle”. Further, one skilled in art of coating will realize that the process for coating the liquid pharmaceutical carrier of matrix particle or the PUFA matrix or carrier article can be used for the initial loading of the PUFA material or liquid pharmaceutical material onto the porous matrix or solid carrier particle.

The term “liquid pharmaceutical” or “pharmaceutical liquid” refers to any pharmaceutically active ingredient existing in a physically liquid form, including in the form of an emulsion, a suspension, a dispersion, an oil or a solution.

The pharmaceutical particles formulated by the claimed processes are suitable for delivery to mammals by a variety of routes of administration including, for example, oral, inhalable, transdermal, parenteral, buccal, nasal, vaginal, rectal, sub-lingual, ocular, periodontal, implantation, or topical.

In a second embodiment the process of the invention relating to coating PUFA matrix or carrier particles or liquid pharmaceutical matrix or carrier particles can be practiced by repeating steps (a)-(c) at least once wherein the liquid coating material is the same or different or repeating steps (a)-(c) at least once using a liquid coating material which can be the same or different from the first liquid coating material.

In a further embodiment, this invention includes a coated PUFA-containing carrier particle or coated PUFA matrix particle made by the claimed process.

In a further embodiment, this invention includes a coated liquid pharmaceutical-containing carrier particle or coated liquid pharmaceutical matrix particle made by the claimed process.

In a further embodiment, this invention includes a food comprising a coated PUFA-containing carrier particle or coated PUFA matrix particle made by the claimed process, or a food comprising a coated liquid pharmaceutical-containing carrier particle or coated liquid pharmaceutical matrix particle made by the claimed process.

In a further embodiment, this invention includes a nutritional supplement comprising a coated PUFA-containing carrier particle or coated PUFA matrix particle made by the claimed process, or a nutritional supplement comprising a coated liquid pharmaceutical-containing carrier particle or coated liquid pharmaceutical matrix particle made by the claimed process

In a further embodiment, this invention includes a beverage comprising a coated PUFA-containing carrier particle or coated PUFA matrix particle made by the claimed process, or a beverage comprising a coated liquid pharmaceutical-containing carrier particle or coated liquid pharmaceutical matrix particle made by the claimed process

In a further embodiment, this invention includes infant formula comprising a coated PUFA-containing carrier particle or coated PUFA matrix particle made by the claimed process, or infant formula comprising a coated liquid pharmaceutical-containing carrier particle or coated liquid pharmaceutical matrix particle made by the claimed process

In a further embodiment, this invention includes a pet food comprising a coated PUFA-containing carrier particle or coated PUFA matrix particle made by the claimed process, or a pet food comprising a coated liquid pharmaceutcal-containing carrier particle or coated liquid pharmaceutical matrix particle made by the claimed process

In a further embodiment, this invention includes animal feed comprising a coated PUFA-containing carrier particle or coated PUFA matrix particle made by the claimed process, or an animal feed comprising a coated liquid pharmaceutical-containing carrier particle or coated liquid pharmaceutical matrix particle made by the claimed process

In a further embodiment, this invention includes the use of a coated PUFA-containing carrier particle or coated PUFA matrix particle made by the claimed process in producing a product selected from the group consisting of a food, nutritional supplement beverage, infant formula, a dairy product, a pet food or animal feed.

In a further embodiment, this invention includes the use of a coated liquid pharmaceutical-containing carrier particle or coated liquid pharmaceutical matrix particle made by the claimed process in producing a product selected from the group consisting of a food, nutritional supplement beverage, infant formula, a dairy product, a pet food or animal feed.

### BRIEF DESCRIPTION OF THE FIGURES

FIG. 1 is a schematic diagram of a portion of the apparatus in accordance with the present invention.

FIG. 2 is a cut away, expanded, cross-sectional view of a portion of the apparatus show in FIG. 1.

FIG. 3 depicts an alternate configuration of the apparatus shown in FIGS. 1 and 2.

### DETAILED DESCRIPTION OF THE INVENTION

All patents, patent applications and publications referred to herein are incorporated by reference in their entirety.

In the context of this disclosure, a number of terms shall be utilized.

The term “polyunsaturated fatty acid” or “PUFA” as used herein refers to fatty acids that contain two or more double bounds. There are two main families of PUFAs, the omega-3 and omega-6 families. The omega-3 fatty acids are those with their endmost double bond 3 carbons from their methyl end. The omega-6 fatty acids are those with their endmost double bond 6 carbon atoms from their methyl end.

Humans cannot efficiently synthesize fatty acids with double bonds located at position 6 or lower in the hydrocarbon chain (when designating position with reference to the methyl end of the fatty acyl chain), e.g., linoleic and linolenic acids. As such, these PUFAs are considered to be “essential fatty acids” in the diet. Fatty acids synthesized from linoleic and linolenic acids, such as arachidonic and eicosapentaenoic and docosahexaenoic acids, are by analogy also essential fatty acids and must be obtained via the diet. All essential fatty acids are polyunsaturated.

The term “PUFA-containing carrier particle” or “pharmaceutical-containing carrier particle” refers to any carrier particle onto which at least one PUFA or pharmaceutical liquid has been adsorbed or at least one PUFA or pharmaceutical liquid has been loaded.

The term “PUFA matrix particle” or “pharmaceutical matrix particle” refers to any type of matrix particle including but not limited to multicore particles, core shell particles, capsules, etc. which contains or incorporates at least one PUFA, or one pharmaceutically active liquid, either alone or in combination with other components at the time that the particle or capsule is formed. Such matrix particles can be made using physical or chemical techniques including, but not limited to spray drying, freeze drying, spinning disc, co-extrusion, spray chilling or spray cooling, liposome entrapment, inclusion complexation, centrifugal extrusion, and rotational suspension separation.

The spray-drying process typically uses a two-nozzle (internal or external mix) assembly, allowing air from an annular geometry to atomize and implode the issuing liquid stream to form fine particles carrying the coated product in a dispersed state. With high particle-specific surface areas, heat from the drying chamber flash-evaporates the solvent or aqueous media, rendering powder capsules that are cyclone-collected into a holding chamber. Some spray-drying operations use rotary atomizers that spin at up to 50,000 rpm.

The freeze-drying process refers to the rapid freezing of a material at low temperature followed by rapid dehydration by sublimation in a high vacuum. This method is used to preserve biological specimens or to concentrate macromolecules with little or no loss of activity. This process is also called “lyophilization”.

The spray chilling or spray cooling process involves mixing the material to be coated with the carrier and atomizing by cooling or chilling air as opposed to heated air in spray drying. Spray chilling is usually used for ferrous sulfate, vitamin, mineral, or acidulent coating. Frozen liquids, heat-sensitive materials and those not soluble in the usual solvents can be coated in this manner. These materials are then released as the wall material is melted. Applications of spray chilling include dry soup mixes, foods with high fat contents, and bakery products.

The spinning disc method, similar to the spray-drying process, uses an emulsion or suspension containing the material to be coated, prepared with a solution or melt of the coating material. The emulsion or suspension is fed to the disc surface and forms a thin wetted layer that, as the disc rotates, breaks up into airborne droplets from surface tension forces that induce thermodynamic instabilities. Resulting capsules are typically spherical. Because the emulsion or suspension is not extruded through orifices, this technique permits use of a higher viscosity shell material and allows higher loading of the material in the shell. The process also offers a broad range of particle sizes with controlled distribution.

Coextrusion coating methods can create fibers containing the active ingredient within fluid, high-viscosity, glassy sugars and carbohydrates. These fibers can be chopped to create microcylinders. When the viscosity is low and the surface tension of the fluid is high, these extrudates would thermodynamically break up into tiny droplets, creating microcapsules.

The typical extrusion systems use stationary nozzle coextrusion, centrifugal coextrusion, or submerged nozzle coextrusion. All these processes involve concentric nozzles, which pump the core material through the inner nozzle while the shell (coating material) formulation is pumped through the annulus, allowing true “core-shell” morphologies. As the liquid stream exits the nozzle, local disturbances, such as induced vibration or gravitational, centrifugal or drag force, control particle size. Typical capsules produced by coextrusion range from 100 micrometers to 6 mm, or about the size of a human egg to the size of a pencil eraser.

Fluidized bed coating involves suspending solid particles in a temperature and humidity controlled chamber of upwardly moving air or other fluidizing gas where the coating material is atomized. The amount of material that coats the particles is dependent on the length of time that the particles are in the chamber.

Liposomes have been used as delivery and carrier systems by coating various compounds in the aqueous layer of the liposome. Phospholipids make up the outer layer or layers of liposomes. The hydrophilic portion of the lipids is oriented towards the aqueous phase and the hydrophobic groups associate with the hydrophobic ones of other lipid molecules. Folding of the lipid sheet into a spherical shape forms a very stable capsule due to there being no interaction of the hydrophobic regions of the lipids with water. Aqueous or lipid-soluble materials, but not both, are entrapped in these membranes. Liposomes can range from a few nanometers to a micron. Liposomes are made by three different procedures. The lipid formulation is mixed with a solvent system such as 2:1 chloroform:methanol. The volume of solvent is decreased and the film of lipids/solvent is then redispersed in an aqueous phase. This step forms the liposomes and it can be performed in different ways including physical, two-phase, and detergent solubilization. The liposomes are then recovered from the water.

Coacervation involves forming microcapsular shells by ionic interaction between two ionic polymers, typically a polyanion (such as acacia gum) and a polycation (such as gelatin). The concept of gelation as a coating method involves using a technique such as cooling, crosslinking, or a chemical reaction to form gelled microspheres or microcapsules. For example, reacting sodium alginate with calcium chloride forms the insoluble calcium alginate.

Inclusion complexation involves the use of a material such as β-cyclodextrin, in which the center is hydrophobic while the outer surface is hydrophilic due to its seven glucose units linked in a β-1,4 configuration. In the center of the cyclodextrin, water molecules are replaced by less polar molecules. The complex then precipitates out of solution. In the case of β-cyclodextrin, only water can serve as the suspension medium. The precipitate is recovered and dried by conventional means.

Rotational or centrifugal suspension separation involves mixing the core and wall materials and then adding to a rotating disc. The core materials then leave the disc with a coating of residual liquid. The capsules are then dried or chilled after removal from the disc. The whole process can take between a few seconds to minutes.

The term “coating” as used herein refers to adherence, adsorption, loading and/or incorporation, to some extent, of at least one liquid coating material onto and/or into a PUFA matrix or carrier particle or liquid pharmaceutical-containing matrix or carrier particle. This coating liquid may remain in the liquid state, or be chilled to solidify or evaporated to leave its solute as a solid coating residue. The coating material on the pharmaceutical or PUFA particle may be of any thickness; it need not necessarily be uniform on the surface of the particle, nor is the entire surface of the particle necessarily covered. As used herein, the term coating includes the concept of encapsulation, but does not necessarily imply that the coated particle has been encapsulated. The term “dry coating” as used herein refers to an aspect of the coating process wherein the particle to be coated is coated in its dry form, the process does not require dispersing the particles in a continuous liquid phase prior to coating, and at the conclusion of the process the particle has no substantial gain in moisture level relative to its uncoated form. The terms “coating” and “dry coating” are used interchangeably herein. As used herein, the term coating does not necessarily imply that the coated particle has been protected from oxidation or diffusion of volatile materials through the coating.

The term “size” as used herein refers to the longest diameter or longest axis of the particle being coated. Throughout the disclosure, the letter “d” or “D” denotes diameter of the particle.

The term “moisture level” as used herein refers to the amount of moisture, for example water or solvent, that is present in the particles before or after coating.

The term “oxidation” as used herein refers to the process wherein the atoms in an element lose electrons thereby making it more electropositive. The valence of the element is correspondingly increased resulting in destruction of fat soluble vitamins, loss of natural colors, decrease or change in aroma and flavor, and creation of toxic metabolites.

The term “volatile” as used herein refers to a compound or material that is readily vaporizable at a relatively low temperature, i.e., it evaporates rapidly. “Volatiles” may refer, for example, to the aroma volatiles within foods, to volatiles in the environment that may diffuse into foods and cause an “off” taste or smell, or to water moisture in gaseous form.

This invention can be practiced using any number of liquid coating materials, general examples of which comprise a starch, gelatin, a natural color, a synthetic color, a sugar, a cellulose, a biodegradable polymer, a biodegradable oligomer, an emulsifying wax, a fat, a wax, a phospholipid, a shellac, a flavoring agent, a moisture barrier, a taste-masking agent, an odor-masking agent, a shelf-life extending agent, a lipid, a protein, cellulose derivatives, alginate, chitosan, surfactants or other wetting agents, carbohydrates, natural or synthetic polymers, or a mineral. Thus, the term “liquid coating material” as used herein includes, but is not limited to, materials that exist as liquids at room temperature as well as those that are solids at room temperature, but have been formulated or dissolved into a liquid state, through the use of solvents or other formulating ingredients, during the coating process. Many liquid coating materials can be used in the process of the invention. In the context of the invention, the term “liquid” refers to the physical state of the coating material as it is applied to the particle. The finally-coated particle, when the particle is at the temperature and other conditions for delivery, may comprise a coating material in either a solid or liquid state. Coating materials include a starch, gelatin, a natural food color, a synthetic food color, a sugar, a cellulose, a biodegradable polymer, a biodegradable oligomer, an emulsifying wax, a shellac, a flavoring agent, a moisture barrier, a taste-masking agent, an odor-masking agent, hydrophobicity or hydrophilicity agents, a shelf-life extending agent, a lipid, a protein, or a mineral. Specific coating ingredients can include, for example, ethyl cellulose, methyl cellulose, hydroxypropylcellulose, polyvinylpyrolidone, polyethylene, Aquateric, Eudragit™ (including any commercial grade or formulations), acrylic coatings, Surelease™, bubble gum flavor, cherry flavor, grape flavor, sodium lauryl sulfate, sodium docusate, poly lactic acid, poly lactide glycolic acid, cellulose acetate pthalate. Further, the following materials comprise suitable coating materials for certain applications, including as diluents: lactose, microcrystalline cellose, mannitol, dicalcium phosphate, starch, dextrates, sucrose; and as disintegrants: croscarmellose sodium, sodium starch glycolate, starch; and as binders: hydroxypropyl cellulose, hydroxypyroylmethylcellulose, povidone, methyl cellulose; and as glidants/lubricants: silicon dioxide, stearic acid, a hydrocolloid, a monosaccharide, a disaccharide, an oligosaccharide, a polysaccharide, a surface modifying agent, a sugar alcohol, a poly-ol, a flow aid, an interparticle force control agent, magnesium stearate, talc, sodium stearyl fumarate; and as surfactants: Tween 80; polysorbate, polyethylene glycol 400, Poloxamer®, glycol 3350, sodium lauryl sulfate (SLS), lecithin, oleic acid, polyoxyethylene alkyl ethers, Cremophor EL, Cremophor RH, polyoxyethlene stearates, sorbitan fatty acid ester; and as additional coating ingredients in the coating material, for example; hydroxypropyl cellulose, hydroxypropylmethylcellulose, titanium dioxide, colors, polyethylene glycols, triethyl citrate, triacetin, dibutyl sebacate and polymethacrylates.

The coating material can specifically be a dispersion of one or more compounds. For example, a coating dispersion may contain a polymer such as ethylcellulose and a plasticizer such as triethyl citrate dissolved in a suitable solvent and talc added as an antitackifier. Solvents that can be used in the process include water, acetone, ethanol, methanol, ethyl acetate, isopropyl alcohol, methyl acetate, n-propanol, ketones, toluene and methylene chloride, for example. A dispersion is defined herein as a two-phase system of which one phase consists of finely divided particles (often in the colloidal size range) distributed throughout a bulk substance, the particles being the disperse or internal phase and the bulk substance the continuous or external phase. Under natural conditions the distribution is seldom uniform, but under controlled conditions the uniformity can be increased by addition of wetting or dispersing agents (surfactants) such as a fatty acid. Examples of dispersions include liquid/liquid (emulsion) and solid/liquid (paint).

PUFA-containing carrier particles or PUFA matrix particles, or liquid pharmaceutical-containing carrier particles or a liquid pharmaceutical matrix particles, can be coated with a combination of liquid coating materials to enhance oxidative stability, protect efficacy and increase shelf life. In addition, unique combinations of flavors, surface modifiers, colors, aromas, etc. can be coated onto the particles. Multiple coatings thus applied can lead to uniquely tailored carrier particle or matrix particle with desired colors, flavorings and freshness aspects; each coating having the ability to retain its original integrity and function, in that there is minimal “mixing” of subsequent layers which are applied to the particles. Another advantage is that the moisture level of the coated materials is substantially the same as the moisture level of the uncoated materials.

Additionally, such particles can be further coated multiple times with the same liquid coating material which is the same or different from the subsequent liquid coating materials, enabling the claimed process to yield particles having particularly controlled thickness of the coating material. Such particles that are coated multiple times with the same liquid coating material can be coated in a successive, batchwise process. It is also possible to provide multiple coatings to a particle by delivering the output of a first apparatus to the feed of a second apparatus in a successive, batchwise process.

There are several benefits of the instant process. It is believed that the process of the instant invention is more cost efficient than currently conducted coating processes, which commonly depend upon spray drying techniques. Further, in one particularly important aspect, the instant process has the flexibility to be operated as a successive, batchwise process. Further, overall particle quality appears to be improved since this is a dry coating process, wherein the liquid coating and drying step occur during the same pass of the food particle through the apparatus of the invention. Overall particle quality is also improved in that the particles that have been coated with the instant process have been observed to retain their morphology, structural integrity and particle size throughout the process. And importantly, the starting moisture level of the coated particles is substantially unchanged during the process. It is desirable that the process yields final coated particles that have not lost moisture and appear too dry, or have taken on additional moisture and become damp, soggy or agglomerated.

The flexibility which is inherent in the operation of the apparatus and process of the invention can result in production of high quality coated PUFA-containing particles or PUFA matrix particles, and high quality liquid pharmaceutical-containing particles or liquid pharmaceutical matrix particles having carefully-controlled and unique characteristics. For example, concentration values of the coating liquid, flow rates of the solid particle feed and the liquid coating feed, ratios of liquid feeds to solid feeds, and temperature and velocity of the gas streams can all be easily varied to yield such coated particles with particular desired characteristics.

The size of the coated carrier particle or matrix particle should not exceed 20.0 mm. The lower limit on size will depend on the PUFA or pharmaceutical liquid being coated, intended use of the product, storage conditions, type of liquid coating material, etc.

The carrier particle when food use is intended for the coated particle can be selected from the group consisting of a protein, fumed silica, titanium dioxide and calcium carbonate, a carbohydrate, a food particle, minerals, salts, antioxidants and lipids. Examples of carrier particles also include, but are not limited to, lactose, modified lactose, corn syrup solids, maltodextrin, starch granules, cellulose and cellulose derivatives, soy cotyledon fiber, spray-dried or freeze dried food particles such as milk powder, instant beverage powders, infant formula powders, creamers, etc.

Examples of suitable food particles which can be used to practice the process of the invention include, but are not limited to, a spray-dried food particle, a freeze-dried food particle, a cereal food, a snack food, a baked good, an extruded food, a fried food, a health food, a dairy food, a pet food or animal feed.

Suitable liquid coating materials will be those which can be used safely in any food or pharmaceutical application such as in any food, nutritional supplement, beverage, infant formula and the like. Applications intended for human consumption should generally utilize materials that are generally recognized as safe (“GRAS”). If the intended application is for incorporation into a pet food or animal feed, then other liquid coating or coating materials may be suitable. For example, some materials recognized as GRAS include but are not limited to the following: polysaccharides/hydrocolloids such as starch, agar/agarose, pectin/polypectate, carrageenan and other gums; proteins such as gelatin, casein, zein, soy and albumin; fats and fatty acids such as mono-, di-, and triglycerides, lauric, capric, palmitic and stearic acid and their salts; cellulosic derivatives; hydrophilic and lipophilic waxes such as shellac, polyethylene glycol, carnauba wax or beeswax; sugar derivatives, etc. Further examples of such liquid coating materials include, but are not limited to, a sweetening agent, a food flavoring agent or enhancer, a food color, a food aroma agent, an anti-caking agent, an humectant, an antimicrobial agent, an antioxidant, a surface modifying agent, a carbohydrate, a protein, a lipid, a mineral, a nutritional supplementing agent, a pharmaceutical, or mixtures thereof.

Non-limiting further examples of coating materials include starch, gelatin, natural food color, a synthetic food color, a sugar, a cellulose, a biodegradable polymer, a biodegradable oligomer, an emulsifying wax, a shellac, a flavoring agent, a moisture barrier, a taste-masking agent, an odor-masking agent, hydrophobicity or hydrophilicity agents, a shelf-life extending agent, a lipid, a protein, or a mineral. Specific coating ingredients can include, for example, ethyl cellulose, methyl cellulose, hydroxypropylcellulose, polyvinylpyrolidone, polyethylene, Aquateric, Eudragit™ (including any commercial grade or formulations), acrylic coatings, Surelease™, bubble gum flavor, cherry flavor, grape flavor, sodium lauryl sulfate, sodium docusate, poly lactic acid, poly lactide glycolic acid, cellulose acetate pthalate. Further, the following materials comprise suitable coating materials for certain applications, including as diluents: lactose, microcrystalline cellose, mannitol, dicalcium phosphate, starch, dextrates, sucrose; and as disintegrants: croscarmellose sodium, sodium starch glycolate, starch; and as binders: hydroxypropyl cellulose, hydroxypyroylmethylcellulose, povidone, methyl cellulose; and as glidants/lubricants: silicon dioxide, stearic acid, a hydrocolloid, a monosaccharide, a disaccharide, an oligosaccharide, a polysaccharide, a surface modifying agent, a sugar alcohol, a poly-ol, a flow aid, an interparticle force control agent, magnesium stearate, talc, sodium stearyl fumarate; and as surfactants: sodium lauryl sulfate, Tween 80; Poloxamer®; and as coating ingredients: hydroxypropyl cellulose, hydroxypropylmethylcellulose, titanium dioxide, colors, polyethylene glycols, triethyl citrate, triacetin, dibutyl sebacate and polymethacrylates.

Examples of sweetening agents include, but are not limited to, sugar substitutes such as saccharin, cyclamate, monellin, thaumatins, curculin, miraculin, stevioside, phyllodulcin, glycyrrhizin, nitroanilines, dihydrochalcones, dulcin, suosan, guanidines, oximes, oxathiazinone dioxides, aspartame, alitame, and the like. There can also be mentioned monosaccharides and oligosaccharides. Examples of monosaccharides include, but are not limited to, galactose, fructose, glucose, sorbose, gatose, tagatose and xylose. As oligosaccharides there can be entioned, sucrose, lactose, lactulose, maltose, isomaltose, maltulose, accharose and trehalose. Other sweetening agents that can also be used include, but are not limited to, high fructose corn syrup. Sugar alcohols, including but not limited to sorbitol, manitol, xylitol, erythritol and the like can also be used.

Examples of food flavoring agents or enhancers include, but are not limited to, monosodium glutamate, maltol, 5′-mononucleotides, such as inosine, and the like.

Examples of food colors include, but are not limited to, tartrazine, riboflavin, curcumin, zeaxanthin, β-carotene, bixin, lycopene, canthaxanthin, astaxanthin, β-apo-8′-carotenal, carmoisine, amaranth, Ponceau 4R (E124), Carmine (E120), anthocyanidin, erythrosine, Red 2G, Indigo Carmine (E132), Patent Blue V (E131), Brilliant blue, chlorophyll, chlorophyllin copper complex, Green S (E142), Black BN (E151), and the like.

Examples of food aroma agents include, but are not limited to, carbonyl compounds, pyranones, furanones, thiols, thioethers, di- and trisulfides, thiophenes, thiazoles, pyrroles, pyridines, pyrazines, phenols, alcohols, hydrocarbons, esters, lactones, terpenes, volatile sulfur compounds and the like.

Examples of an anti-caking agents include, but are not limited to, sodium, potassium, calcium hexacyanoferrate (II), calcium silicate, magnesium silicate, tricalcium phosphate, magnesium carbonate and the like.

Examples of humectants include, but are not limited to, 1,2-propanediol, glycerol, manitol, sorbitol and the like.

Examples of antimicrobial agents include, but are not limited to, benzoic acid, PHB esters, sorbic acid, propionic acid, acetic acid, sodium sulfate and sodium metabisulfite, diethyl pyrocarbonate, ethylene oxide, propylene oxide, nitrite, nitrate, antibiotics, diphenyl, o-phenylphenol, thiabendazole and the like.

Examples of antioxidant agents include, but are not limited to, tocopherols, 2,6-di-tert-butyl-p-cresol (BHT), tert-butyl-4-hydroxyanisole (BHA), propylgallate, octylgallate, dodecylgallate, ethoxyquin, ascorbyl palmitate, ascorbic acid and the like.

Examples of surface modifying agents include, but are not limited to, mono-, diaglycerides and derivatives, sugar esters, sorbitan fatty acid esters, polyoxyethylene sorbitan esters, stearyl-2-lactylate and the like.

Examples of nutritional supplementing agents include, but are not limited to, vitamins group consisting of fat soluble vitamins group consisting of retinol (vit A), calciferol (vit D), tocopherol (vit E), phytomenadione (vit K1), water soluble vitamins group consisting of thiamine (vit B1), riboflavin (vit B2), pyridoxine (vit B6), nicotinamide (niacin), pantothenic acid, biotin, folic acid, cyanocobalamin (vit B12), ascorbic acid (vit C), polyunsaturated fatty acids (PUFA), and the like.

Other carbohydrates which can be used in a liquid coating material include polysaccharides such as agar, alginates, carrageenans, furcellaran, gum arabic, gum ghatti, gum tragacanth, karaya gum, guaran gum, locust bean gum, tamarind flour, arabinogalactan, pectin, starch, modified starches, dextrins, cellulose, cellulose derivatives, hemicelluloses, xanthan gum, scleroglucan, dextran, polyvinyl pyrrolidone and the like.

Examples of lipids include, but are not limited to, saturated and unsaturated fatty acids, mono- and diacylglycerols triacylglycerols, phospholipids, glycolipids, phosphatidyl derivatives, glycerolglycolipids, sphingolipids, lipoproteins, diol lipids, waxes, cutin and the like.

Examples of minerals include, but are not limited to, salts of sodium, potassium, magnesium, calcium, chloride, phosphate, iron, copper, zinc, manganese, cobalt, vanadium, chromium, selenium, molybdenum, nickel, boron, silica, silicon, fluorine, iodine, arsenic and the like.

Any of the liquid coating materials discussed herein can be used in practicing the process of the invention. In addition, there can be used a shelf-life extending agent such as an oxygen barrier and a flavor retaining agent such as a volatile barrier.

Any PUFA can be used to practice the invention. There can be mentioned γ-linolenic acid (GLA), dihomo-γ-linolenic acid, arachidonic acid (ARA), docosahexaenoic acid (DHA) and/or eicosapentaenoic acid (EPA).

Examples of foods into which an coated PUFA-containing carrier particle or coated PUFA matrix particle, or pharmaceutical-containing carrier particle or pharmaceutical matrix particle can be incorporated include, but are not limited to, a cereal food product, a snack food product, a baked good product, a fried food product, a health food product, infant formula, beverages, a dairy product, a nutritional supplement, a pet food product, and animal feed.

A cereal food product is a food product derived from the processing of a cereal grain. A cereal grain includes any plant from the grass family that yields an edible grain (seed). The most popular grains are barley, corn, millet, oats, quinoa, rice, rye, sorghum, triticale, wheat and wild rice. Examples of a cereal food product include, but are not limited to, whole grain, crushed grain, grits, flour, bran, germ, breakfast cereals, extruded foods, pastas, and the like.

A baked good product comprises any of the cereal food products mentioned above and has been baked or processed in a manner comparable to baking, i.e., to dry or harden by subjecting to heat. Examples of a baked good product include, but are not limited to breadcrumbs, baked snacks, mini-biscuits, mini-crackers, mini-cookies, and mini-pretzels.

A snack food product comprises any of the above or below described food products.

A fried food product comprises any of the above or below described food products that has been fried.

A health food product is any food product that imparts a health benefit. Many oilseed-derived food products may be considered as health foods. There can be mentioned soybeans, flax seed, sesame seed, pumpkin seeds, sunflower seeds, or food products processed from these seeds or which are incorporated into foods. For example, soy nuggets and soy nuts can be mentioned. In addition to oilseed-derived food products, fruit-derived food products can be mentioned such as fruit bits, dried berries, and the like.

A beverage is any drinkable liquid. For example, there can be mentioned non-carbonated drinks; carbonated drinks; fruit juices, fresh, frozen, canned or concentrate; still or sparkling water; flavored or plain milk drinks, etc. Adult and infant nutritional formulas are well known in the art and commercially available (e.g., Similac®, Ensure®, Jevity®, and Alimentum® from Ross Products Division, Abbott Laboratories).

A dairy product is a product derived from milk. These products include, but are not limited to, whole milk, skim milk, fermented milk products such as yogurt or sour milk, cream, butter, condensed milk, dehydrated milk, coffee whitener, ice cream, cheese, whey products, and lactose.

Infant formulas are liquids or reconstituted powders fed to infants and young children. They serve as substitutes for human milk. Infant formulas have a special role to play in the diets of infants because they are often the only source of nutrients for infants. Although breast-feeding is considered the best nourishment for infants, infant formula is a close enough second that babies not only survive but also thrive. Infant formula compositions are becoming increasingly close to the composition of breast milk.

A pet food product is a product intended to be fed to a pet such as a dog, cat, bird, reptile, fish, rodent and the like. These products can include the cereal and health food products above, as well as meat and meat byproducts, grass and hay products, including but not limited to alfalfa, timothy, oat or brome grass and the like.

Animal feed is a product intended to be fed to animals such as turkeys, chickens, cattle and swine and the like. As with the pet foods above, these products can include cereal and health food products, meat and meat byproducts, and grass and hay products as listed above.

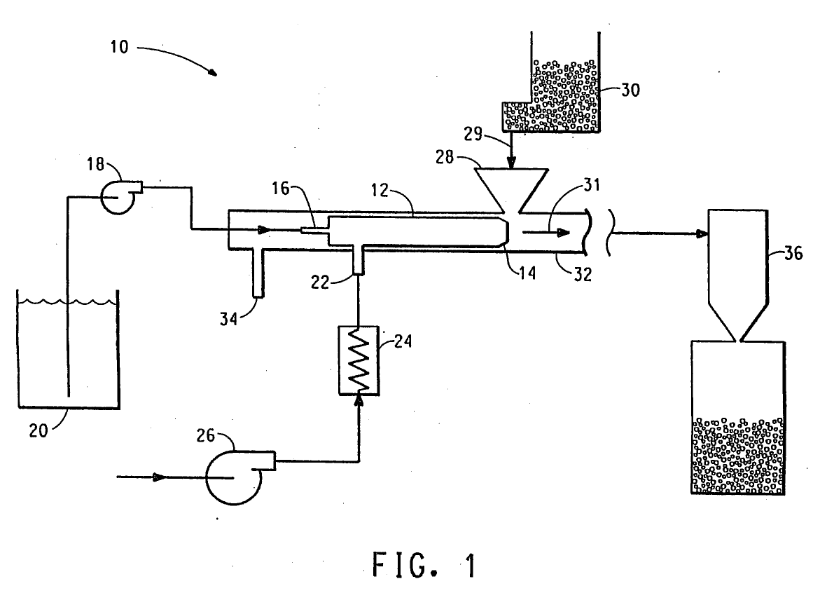

The apparatus used to practice the process of this invention is generally as described in commonly-owned PCT application WO 97/07879 which is discussed above. An apparatus according to the present invention is shown generally at 10 in FIG. 1.

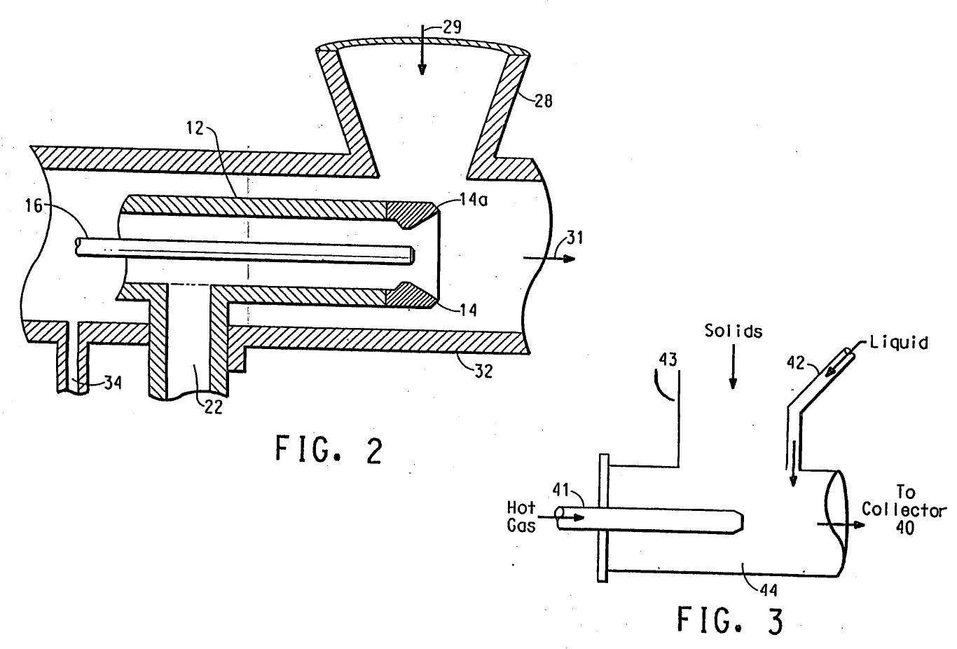

The apparatus of the present invention comprises a first chamber, shown at 12 in FIGS. 1 and 2. A flow restrictor 14 is disposed at one end of the first chamber. The flow restrictor is typically disposed at the downstream end of the first chamber, as shown in FIGS. 1 and 2. Flow restrictor 14 has an outlet end 14a, as shown in the detailed view of FIG. 2.

Although the flow restrictor is shown as a different element from the first chamber, it may be formed integrally therewith, if desired. The flow restrictor of the present invention may have various configurations, as long as it serves to restrict flow and thereby increase the pressure of the fluid passing through it. Typically, the flow restrictor of the present invention is a nozzle.

A first, or liquid, inlet line 16 as shown in FIGS. 1 and 2 is disposed in fluid communication with the first chamber for metering a liquid composition into the chamber. Liquid inlet line 16 meters the liquid composition into first chamber 12 through the outlet of flow restrictor 14, and preferably in the center of the flow restrictor when viewed along the axial length thereof. The liquid composition is metered through liquid inlet line 16 by a metering pump 18 from a storage container 20 containing the liquid composition as shown in FIG. 1.

The liquid coating composition may be wherein a material which is used as the coating material is dissolved in a liquid, or a slurry, a dispersion, or an emulsion where a material which is used as the coating material is undissolved in a liquid. Alternatively, the liquid coating composition may be a melt, which is used as the coating material. By melt is meant any substance at a temperature at or above it melting point, but below its boiling point. In any of these cases, the liquid composition may include components other than the coating material. It should be noted that when the liquid composition is a melt, storage container 20 must be heated to a temperature above the melt temperature of the liquid composition in order to maintain the liquid composition in melt form. Further, the coating material can be a dispersion of one or more compounds. For example, a coating dispersion may contain a polymer such as ethylcellulose and a plasticizer such as triethyl citrate dissolved in a suitable solvent and talc added as an antitackifier. Solvents that can be used in the process include water, acetone, ethanol, methanol, ethyl acetate, isopropyl alcohol, methyl acetate, n-propanol, ketones, toluene and methylene chloride, for example. A dispersion is defined herein as a two-phase system of which one phase consists of finely divided particles (often in the colloidal size range) distributed throughout a bulk substance, the particles being the disperse or internal phase and the bulk substance the continuous or external phase. Under natural conditions the distribution is seldom uniform, but under controlled conditions the uniformity can be increased by addition of wetting or dispersing agents (surfactants) such as a fatty acid. Examples of dispersions include liquid/liquid (emulsion) and solid/liquid (paint).

The apparatus for coating a PUFA carrier or matrix particle or a liquid pharmaceutical matrix or carrier particle further includes a second, or gas, inlet line 22 disposed in fluid communication with the first chamber as shown in FIGS. 1 and 2. Generally, the gas inlet line should be disposed in fluid communication with the first chamber upstream of the flow restrictor. Gas inlet line 22 injects a first gas stream through the flow restrictor to create turbulent flow of the gas stream. The turbulence subjects the liquid composition to shear forces that atomize the liquid composition.

The first gas stream should have a stagnation pressure sufficient to accelerate the gas to at least one-half the velocity of sound, or greater, prior to entering the flow restrictor to ensure that a turbulent flow of gas of sufficient intensity will be formed at the outlet of the flow restrictor. The velocity of sound for a particular gas stream, e.g., air or nitrogen, will be dependent on the temperature of the gas stream. This is expressed by the equation for the speed of sound, C:
C=\sqrt{kgRT}
where:
   k=ratio of specific heats for the gas
   g=acceleration of gravity
   R=universal gas constant
   T=absolute temperature of the gas

Thus, the acceleration of the first gas stream is dependent on the temperature of the gas stream.

As noted above, it is the pressurized gas that causes the atomization of the liquid composition. The pressure of the liquid composition in the liquid inlet line just needs to be enough to overcome the system pressure of the gas stream. It is preferable that the liquid inlet line has an extended axial length upstream of the flow restrictor 14. If the liquid inlet line is too short, the flow restrictor becomes plugged.

The apparatus of the present invention also comprises means disposed in the second inlet line and upstream of the flow restrictor for optionally heating the first gas stream prior to injection through the flow restrictor. Preferably, the heating means comprises a heater 24 as shown in FIG. 1. Alternatively, the heating means may comprise a heat exchanger, a resistance heater, an electric heater, or any type of heating device. Heater 24 is disposed in second inlet line 22. A pump 26 as shown in FIG. 1 conveys the first gas stream through heater 24 and into first chamber 12. When a melt is used as the coating material, the gas stream should be heated to a temperature around the melt temperature of the liquid composition to ensure solidification of the melt on the particles. As also noted above for the apparatus, when using a melt, it is also helpful if auxiliary heat is provided to the first inlet line, which supplies the melt prior to injection, to prevent pluggage of the line.

The apparatus of the present invention further includes a second chamber 32 surrounding the first chamber as shown in FIGS. 1 and 2. The second chamber encloses the turbulent flow of gas. The apparatus of the present invention further includes a hopper 28 as shown in FIGS. 1 and 2. Hopper 28 introduces a particle into the region of the second chamber 32 in which turbulent flow of the gas has been created. It is preferable that the outlet end of the flow restrictor is positioned in the first chamber beneath the hopper at the centerline of the hopper (i.e., the region in which the turbulent flow of gas is created). This ensures that the particles are introduced directly into the turbulent flow of gas. This is important because, as noted above, the turbulence subjects the liquid composition to shear forces that atomize the liquid composition. It also increases operability by providing a configuration for feeding the particles most easily. In addition, the shear forces disperse and mix the atomized liquid composition with the particles, which allows the particles to be coated within the turbulent flow. Hopper 28 may be fed directly from a storage container 30 as shown by arrow 29 in FIG. 1. The hopper of the present invention may include a metering device for accurately metering the particles at a particular ratio to the liquid feed from liquid inlet line 16 into the zone of turbulence. This metering establishes the level of coating on the particle or coating of the particle. Typically, the hopper of the present invention is open to the atmosphere. When a melt is used, it is preferred that the particles are at ambient temperature because this facilitates solidification of the melt after the melt which is initially at a higher temperature, encapsulates/coats the particle in the zone of turbulent flow.

The apparatus of the present invention further includes an inlet 34 for introducing a second gas stream into the second chamber. The inlet of the second gas stream is preferably positioned at or near the upstream end of second chamber 32. The outlet of second chamber 32 is connected to a collection container, such as that shown at 36 in FIG. 1. The second gas stream acts to reduce any tendency for recirculation within the region of turbulent flow and cools and conveys the coated particles toward the collection container as illustrated by arrow 31 in FIG. 2.

In particular, when a solution, dispersion or slurry is used, the solid of the solution, dispersion or slurry cools between the zone of turbulence and container so that by the time the particle reaches the container, a solid coating comprising the solid of the solution, dispersion or slurry is formed on the particle. When a melt is used, the liquid composition cools within the zone of turbulence so that by the time the particle reaches the container, a solid coating comprising the melt is formed on the particle. The first gas stream, as well as the second gas stream, is vented through the top of collection container 36.

For the configuration as shown in FIGS. 1 and 2, inlet 34 may be connected to a blower, not shown, which supplies the second gas stream to the second chamber. However, the blower and second chamber 32 may be eliminated, and the first gas stream may be used to cool the particles and to convey them to container 36. In this case, the solid from the solution, dispersion or slurry or the melt cools and solidifies on the particle in the atmosphere between the zone of turbulence and the collection container, and the coated particles fall into collection container 36.

It is preferable that the axial length of the region of the second chamber in which the turbulent flow is created is about ten times the diameter of the second chamber. This allows the pressure at the outlet of the flow restrictor to be at a minimum. Particles are fed into second chamber 32 as shown in FIGS. 1 and 2 near the outlet of the flow restrictor, which is preferably positioned at the centerline of the hopper. If the pressure at the outlet is too great, the particles will back flow into the hopper.

The pressure of the second gas stream must be sufficient to assist in conveying the coated particles from the zone of turbulence to the collection zone, but should be at lower than the pressure of the first gas stream. This is because a high relative velocity difference between the first gas stream and the second gas stream is needed to produce a sufficient degree of turbulence to encapsulate/coat the particles.

It should be noted that the process of the present invention may be practiced using the apparatus illustrated in FIGS. 1, 2 and 3, although it should be understood that the process of the present invention is not limited to the illustrated apparatus. Moreover, it should be noted that while one pass, or cycle, of the process of the present invention substantially or completely encapsulate/coats the particle, more than one pass may be used to adhere additional coating material to the particle, depending on the desired thickness of the liquid coating material.

The process comprises the steps of metering a liquid composition into a flow restrictor, such as flow restrictor 14 as shown in FIGS. 1 and 2. As described above for the apparatus, the liquid composition may be a solution, dispersion, slurry, emulsion or melt.

The process of the present invention further comprises injecting a gas stream, for instance from a gas inlet line such as that shown at 22 in FIGS. 1 and 2, through the flow restrictor concurrently with metering the liquid composition into the flow restrictor, to create turbulent flow of gas at the outlet of the flow restrictor. The shear in the zone of turbulence atomizes the liquid composition.

The gas stream is controlled prior to injecting it through the flow restrictor. The gas stream may be heated by a heater, such as heater 24 as shown in FIG. 1. As noted above for the apparatus, when the liquid composition is a solution, dispersion or slurry, the gas stream is heated to a temperature sufficient to vaporize the liquid of the solution, dispersion or slurry and to leave the solid of the solution, dispersion or slurry remaining. When the liquid composition is a melt, the gas stream should be heated to a temperature around the melt temperature of the liquid composition, to keep the liquid composition, and in particular, the melt, in liquid (i.e., melt) form. As also noted above for the apparatus, when using a melt, it is also helpful if auxiliary heat is provided to the first inlet line which supplies the melt prior to injection, to prevent pluggage of the line.

The process of the present invention also comprises the step of adding a PUFA carrier or matrix particle or liquid pharmaceutical carrier or matrix particle to the zone of turbulence concurrently with the metering of the liquid composition and the injection of the gas stream. This mixes the particle with the atomized liquid composition at the zone of turbulence. This mixing at the zone of turbulence coats the particle with the liquid coating material. The particle is preferably metered in order to control the ratio of the particle and the liquid added at the zone of turbulence. This establishes the level of coating of the particle. When a solution, dispersion or slurry is used, the heat from the heated gas stream serves to evaporate the liquid of the solution, dispersion or slurry, leaving the solid of the solution, dispersion or slurry remaining to encapsulate/coat the particle. The mixing at the zone of turbulence then coats the particle with the remaining solid from the solution, dispersion or slurry. When a melt is used, the mixing at the zone of turbulence coats the particle with the melt. Particle sizes should not exceed 20.0 mm.

As noted above, the zone of turbulence is formed by the action of injecting the gas at high pressure through the flow restrictor. As discussed above with respect to the apparatus, it is preferable that the gas stream is accelerated to at least about one-half the velocity of sound prior to injection to ensure that a zone of turbulence of sufficient intensity will be formed at the outlet of the flow restrictor.

The residence time of the particles in the zone of turbulence is determined by the geometry of the first chamber and the amount of gas injected from the gas inlet line. The average residence time of the particle within the zone of turbulence is preferably less than 250 milliseconds. More preferably, the average residence time of the particle within the zone of turbulence is in the range of 25 to 250 milliseconds. Short residence times can be achieved because of the action of the zone of turbulence. The short residence times make the process of the present invention advantageous compared to conventional coating processes because the time, and hence, the cost of coating particles, are reduced. Typically, the particles are fed from a hopper, such as hopper 28 as shown in FIGS. 1 and 2, which is open to the atmosphere. As noted above for the apparatus, when the liquid composition is a melt, it is preferred that the particles be at ambient temperature because this will facilitate solidification of the melt after the melt (which is initially at a higher temperature) coats the particle in the zone of turbulence.

The process of the present invention may further comprise the step of adding another gas stream upstream of the zone of turbulence for cooling and conveying the coated particle. This other gas stream is added through a chamber, such as second chamber 32 as shown in FIGS. 1 and 2. As explained above for the apparatus, the pressure of the second gas stream must be sufficient to assist in conveying the coated particles from the zone of turbulence to the collection container, but should be at lower than the pressure of the first gas stream in order to achieve coating. When a solution, dispersion or slurry is used, the solid of the solution, dispersion or slurry cools and solidifies on the particle in the second chamber between the zone of turbulence and a collection container, such as collection zone 36 as described above. When a melt is used, the melt cools and solidifies on the particle in the second chamber between the zone of turbulence and the collection container. When a second chamber is not included, the solid or the melt cools and solidifies on the particle in the atmosphere between the zone of turbulence and the collection container, and the coated particles fall into the container.

The coating materials are generally liquid in nature, and can be single or multiple chemical compositions. Thus, they may be pure liquids, dispersions including suspensions and emulsions, melted polymers, resins, and the like. These materials generally have viscosities in the 1 to 2,000 centipoise range. Coatings that are applied can be hydrophilic, hydrophobic or amphoteric in nature, depending on their chemical composition. When more than one coating layer is applied, it can be either as another shell adhering to the previous coating layer, or as individual particles on the surface of the material to be coated. These materials may also be reactive so that they cause the material they are coating to increase in viscosity or change to a solid or semi-solid material. So that the coating formed on the selected material is in the range stated above, the coating material should be capable of being molecularly dispersed, so that the coating can grow from the molecular level.

The apparatus as shown in FIGS. 1, 2, and 3 can be used for a number of processes. One such process is that of coating a PUFA-or pharmaceutical-containing particle or PUFA or pharmaceutical matrix particle. In this process, the particle or matrix particle enters the apparatus and the material that will be used to coat the particles is fed into the apparatus through the hopper into the high shear/turbulence zone. The resulting atomized coating material coats the surface of the particle, and it is pneumatically transported through the apparatus. The temperature of the process generally exceeds the boiling point of the solvent (at least by 5° C. in many cases). This ensures rapid evaporation of the solvent from the liquid coated particles, where desired. The coated material is then transported out of the apparatus in a substantially dry state, such that there is substantially no net moisture gain from one end of the process to the other. The moisture content is measured by a Cenco moisture balance operated at 105° C. Thus, the coating and drying of the material is accomplished in a single step. This is important so that the quality of the particle is maintained, as the particles are uniformly coated, the material is not degraded by excessive exposure to relatively high temperatures, and the particles do not agglomerate or stick to the sides of the vessels. Furthermore, the moisture level of the coated particle is substantially the same as the moisture level of the uncoated particle.

A convective drying process is used for removing residual volatiles that result from putting a solution, dispersion, slurry or emulsion coating onto the surface of a particle. The design of the process precludes wet particles from reaching any wall to which they may stick, which improves the cleanliness of the system, and may also include a recycle system which can reduce any interparticle or particle-to-wall sticking that might otherwise occur. This process may be selected from any number of methods, including but not limited to flash drying, pneumatic conveyor drying and spray drying, or combinations thereof. Residence times for drying are generally less than a minute, and preferably in the millisecond time frame.

As shown in FIG. 3, the apparatus of FIGS. 1 and 2 can have an alternate configuration. Solids enter the apparatus through hopper 43. Liquid is added via a liquid inlet tube 42 located at the top of the apparatus, so that the liquid exits into the high shear/turbulence zone. Hot gas enters chamber 44 through nozzle 41. Product outlet from chamber 44 exits to collector 40. This configuration can allow for faster changes of liquid used for coating, and is less expensive to maintain.

### EXAMPLES

The invention is further described by the following Examples, which are provided for illustration and are not to be construed as limiting the scope of the invention.

Coating layers that were produced according to the Examples were calculated as their percent contribution to the mass of the coated particle. Coating levels were determined based on mass balance.

### Example 1

### Preparation of PUFA-Coated Isolated Soy Protein Particles

A preparation of isolated soy protein (“ISP”, Supro 500E, DuPont Protein Technologies, St. Louis, Mo.) was coated with polyunsaturated fatty acid in order to produce PUFA coated protein in a single coating and drying process. The apparatus as shown in FIG. 1 had a mixing chamber 32 mm in diameter and 300 mm in length with a nozzle throat of 10 mm and a central liquid feed tube of 6.5 mm O.D. and 4.8 mm I.D. The apparatus has a single screw metering feeder (AccuRate) or a vibrating feeder (Syntron) for metering the solid particles. A peristaltic pump was fit with 6.5 mm Tygon elastomer tubing for metering the liquid. Supro 500E was used without further treatment and was metered to the system at 990 grams/minute. PUFA-enriched oil (Omega Protein Inc., Houston, Tex.) was at 22° C. and was metered at 21.9 g/min. to the center tube using the peristaltic metering pump. Nitrogen gas was supplied to the nozzle at 414 KPa, and was at 22° C. at the nozzle. The nitrogen gas was used to atomize the PUFA-enriched oil, producing a negative pressure in the mixing zone to induce the addition of the Supro 500E, and provide the heat for evaporating any residual moisture from the Supro 500E. The product of the mixing/drying was collected in a polyester twill bag filter immediately. The product had an oil-coating layer equal to 2.16% of the final mass of the coated particle. There was no gain in residual moisture (as measured by a Cenco moisture balance) when compared to the particles prior to coating. The PUFA oil-coated ISP material retained the dry flowable property of the uncoated ISP starting material.

### Examples 2-7

### Preparation of PUFA-Coated Isolated Soy Protein Particles with Various Levels of PUFA Oil Coating

Additional lots of PUFA oil-coated isolated soy protein particles were prepared using the apparatus and method of Example 1. Differing levels of PUFA oil in the final product were achieved by modifying the operating parameters of the process. The modifications to the process and the amount of PUFA oil in the products thus formed are listed in Table 1. Menhaden oil (OmegaPure, Houston, Tex.) was used as the PUFA oil in Example 7.

TABLE 1

| | Drying | Gas | ISP | PUFA | PUFA oil | PUFA oil |
|---|---|---|---|---|---|---|
| | gas, | nozzle | feed | oil | feed | in finished |
| Ex- | pressure | temp. | rate | feed rate | temp. | particle |
| ample | (Kpa) | (° C.) | (g/min) | (g/min) | (° C.) | (%) |
| 2 | N₂, 414 | 18.5 | 1181 | 158 | 22 | 11.8 |
| 3 | N₂, 414 | 16.2 | 736 | 80.3 | 22 | 9.8 |
| 4 | N₂, 414 | 15.9 | 1004 | 184 | 22 | 15.5 |
| 5 | N₂, 414 | 15.5 | 604 | 235 | 22 | 28.1 |
| 6 | N₂, 414 | 19.5 | 1006 | 92 | 22 | 8.4 |
| 7 | N₂, 414 | 19. | 1104 | 138 | 22 | 11.1 |

PUFA oil coating layers on the isolated soy protein particles ranged from approximately 2% to approximately 30% of the final product.

### Example 8

### PUFA Oil-Coated Isolated Soy Protein Particles Further Coated with a Layer of Sucrose

The PUFA oil-coated isolated soy protein prepared as described in Example 3, above, was used as the solid feed material in the coating process of the invention to produce a PUFA oil-coated particle with a relatively light barrier layer of solid sucrose. The apparatus was as described in Example 1 with the following operational modifications. The nitrogen employed as drying gas had a nozzle temperature of 315° C. The PUFA oil-coated isolated soy protein particles were metered into the apparatus at a rate of 743 g/min. A solution of food grade sucrose (84% w/w in water) was metered into the apparatus at a rate of 25 g/min and at a temperature of 95° C. The dry coated particles were collected as described in Example 1. The resultant particle possessed a first, internal coating layer of PUFA oil and a second, external coating/coating layer of sucrose that constituted 2.8% of the finished product. The solid coating/coating layer on a PUFA material is useful as a barrier against the undesirable effects of oxidation, and it improves the handling characteristics of the PUFA oil-coated particles.

Oxidation rates of the PUFA oil component of the particles may be determined by standard methods available in the art. These include the Active Oxygen Method, method Cd12-57 of the American Oil Chemists' Society (AOCS), and the Oil Stability Index, AOCS method Cd 12b-92.

### Example 9

### PUFA Oil-Coated Isolated Soy Protein Particles Further Coated with a Heavy Layer of Sucrose

The PUFA oil-coated isolated soy protein prepared as described in Example 3, above, was used as the solid feed material in the coating-process of the invention to produce a PUFA oil-coated particle with a heavy layer of solid sucrose. The apparatus was as described in Example 1 with the following operational modifications. The nitrogen employed as drying gas had a nozzle temperature of 319° C. The PUFA oil-coated isolated soy protein particles were metered into the apparatus at a rate of 743 g/min. A solution food grade of sucrose (84% w/w in water) was metered into the apparatus at a rate of 53 g/min and at a temperature of 95° C. The dry coated particles were collected as described in Example 1. The resultant particle possessed a first, internal coating of PUFA oil and a second, external coating of sucrose constituting 8.1% of the finished product. The solid coating on a PUFA material is useful as a barrier against the undesirable effects of oxidation, and it improves the handling characteristics of the PUFA oil-coated particles.

Oxidation rates of the PUFA oil component of the particles may be determined by standard methods available in the art. These include the Active Oxygen Method, method Cd12-57 of the American Oil Chemists' Society (AOCS), and the Oil Stability Index, AOCS method Cd 12b-92.

### Example 10

### PUFA Oil-Coated Isolated Soy Protein Particles Further Coated with a Layer of Protein

The PUFA oil-coated isolated soy protein prepared in Example 7, above, was further coated with casein to provide a barrier to moisture and oxidation to the PUFA material. The apparatus and process was as described in Example 1 with the following operational modifications. The PUFA oil-coated isolated soy protein particles (12.5% PUFA oil coating) were metered into the apparatus at a rate of 300 g/min. A solution of casein (20% w/w in water, prepared from Non Fat Dry Milk Powder Low Heat A Grade, T. C. Jacoby & Co. Inc., St Louis, Mo.) was metered into the apparatus at a rate of 48 g/min and at a temperature of 30° C. The dry coated particles were collected as described in Example 1. The resultant particle possessed a first, internal coating of PUFA oil and a second, external coating of dried casein that constituted 3.1% of the finished product. Such a solid coating on a PUFA material is useful as a barrier to both moisture and oxidation.

### Examples 11-14

### PUFA Oil-Coated Isolated Soy Protein Particles Further Coated with a Various Levels of Protein

Additional lots of isolated soy protein particles coated with both PUFA oil and casein were prepared as generally described in Example 10. All of the products of these Examples possessed a barrier layer of dried casein to protect the PUFA oil that had first been coated onto the isolated soy protein. Differing amounts of casein in the final product were achieved by modifying the operating parameters of the process. The modifications to the process and the amount of casein in the products thus formed are listed in Table 2.

TABLE 2

| | Drying | Gas | Solid | Casein | Casein | Casein |
|---|---|---|---|---|---|---|
| | gas, | nozzle | feed | oil | feed | in finished |
| Ex- | pressure | temp. | rate | feed rate | temp. | particle |
| ample | (Kpa) | (° C.) | (g/min) | (g/min) | (° C.) | (%) |
| 11 | N₂, 414 | 294 | 300 | 48 | 30 | 3.1 |
| 12 | N₂, 414 | 285 | 300 | 48 | 30 | 6.1 |
| 13 | N₂, 414 | 276 | 300 | 47 | 30 | 9.0 |
| 14 | N₂, 414 | 281 | 300 | 48 | 30 | 11.8 |

The casein barrier coating on the PUFA oil-coated isolated soy protein ranged from approximately 3% to 12% of the final product.

### Example 15

### Isolated Soy Protein Particle Multiply Coated with PUFA Oil, Casein, and a High Melting Temperature Fat

The multiply coated isolated soy protein particle produced in Example 13, above, was further processed to provide an additional barrier layer of fat with a high melting temperature. The apparatus and process is as described in Example 1, with the following modifications to the process. Isolated soy protein particles possessing coatings of PUFA oil and casein were prepared as described in Example 13. The particles were metered to the system at 611 g/min. A preparation of high melting temperature fat (Dritex, ACH Food Companies, Cordova, Tenn.) was metered at 120 g/min. to the center tube at 80° C. using the peristaltic metering pump. Nitrogen gas was supplied to the nozzle at 414 KPa, and was at 216° C. at the nozzle. The dry coated particles were collected as described in Example 1. The resultant particle possessed a first internal coating of PUFA oil, a second internal coating casein, and a third, external coating of Dritex that constituted 16.4% of the finished product.

The high melting fat layer provides additional protection against moisture. The effectiveness of the moisture barrier was demonstrated by differential, temperature-dependent dispersal of the protein into water. High melting point fat-coated particles (approximately 1 g) were placed into 150 mL of water in a beaker at room temperature. A portion of the fat-coated particles floated on the surface of the water, while the remainder sank below the surface. The beaker was shaken gently and observed for 5 minutes. No substantial change in the appearance of the water was observed. It was concluded that the oil and soy protein in the particles were protected from the water by the fat barrier because the water did not become cloudy. Similarly, high melting point fat-coated particles were added to water at 90° C. Within a few seconds, the particles dispersed into the water and the water became cloudy and off-white. It was concluded that the protection of the soy protein and PUFA oil from the water by fat was not possible above the melting point of the fat barrier (70° C.). Thus the protein was delivered to the water at a temperature that caused the fat to melt.

### Examples 16-24

### Loading of PUFA Oil onto Solid Carriers

PUFA oil was loaded onto solid carrier particles to produce a series of dry flowable oil products in which the oil was present as a coated liquid. The apparatus and processes were as described in Example 1, above, with operational modifications listed in Table 3. Carrier particles used as the solid feed material in the process included titanium dioxide (TiO₂, 72% w/w slurry of pigment grade material in water, DuPont. Wilmington, Del.), CaCO₃ (Camel-CARB, Genstar Stone Products Co. Hunt Valley, Md.), and fumed silica (Cab-O-Sil EH-5, approximately 600 m₂ surface area per gram, Cabot Corporation, Boston, Mass.). The final composition of the product of each example is given as the percentage that is PUFA oil.

TABLE 3

| | Drying | Gas | | Solid | PUFA oil | PUFA oil | PUFA |
|---|---|---|---|---|---|---|---|
| | gas, | nozzle | Solid | feed | feed | feed | oil in |
| | pressure | temp. | feed | rate | rate | temp. | finished |
| Example | (Kpa) | (° C.) | material | (g/min) | (g/min) | (° C.) | particle (%) |
| 16 | Air, 345 | 20 | TiO₂ | 410 | 25 | 22 | 5.8 |
| 17 | Air, 345 | 20 | TiO₂ | 763 | 74 | 22 | 8.8 |
| 18 | Air, 345 | 20 | TiO₂ | 692 | 143 | 22 | 17.1 |
| 19 | Air, 345 | 21 | CaCO₃ | 604 | 33 | 22 | 5.5 |
| 20 | Air, 345 | 21 | CaCO₃ | 628 | 69 | 22 | 9.9 |
| 21 | Air, 345 | 21 | CaCO₃ | 607 | 78 | 22 | 11.4 |
| 22 | Air, 345 | 21 | CaCO₃ | 599 | 116 | 22 | 16.3 |
| 23 | N₂, 414 | 23 | Cab-O-Sil | 489 | 76 | 22 | 13.5 |
| 24 | N₂, 414 | 22 | Cab-O-Sil | 218 | 134 | 22 | 38.1 |

The results of these Examples demonstrate that PUFA oil can be loaded onto a variety of solid carrier particles, at levels approaching 40% of the final material. The resulting particles behave as if they are dry powders and offer a number of advantages for storage and handling, and for the incorporation of PUFA-containing materials into foods and nutritional products.

### Example 25

### Coating of Lightly Loaded PUFA Particle with a Sucrose Layer

The PUFA-coated fumed silica particle produced in Example 23, above, was further coated to produce a barrier layer of solid sucrose. The apparatus was as described in Example 1 with the following operational modifications. Nitrogen was employed as the drying gas and had a nozzle temperature of 294° C. The PUFA oil-coated Cab-O-Sil particles from Example 23 were metered into the apparatus at a rate of 487 g/min. A solution of food grade sucrose (84% w/w in water) was metered into the apparatus at a rate of 97 g/min and at a temperature of 95° C. The dry coated particles were collected as described in Example 1. The resultant particle possessed a first, internal layer of PUFA oil and a second, external layer of sucrose that constituted 14.4% of the finished product. Such a solid coating on a PUFA material is useful as a barrier to the undesirable effects of oxidation and it improves the handling characteristics of the PUFA oil-coated particles.

### Example 26

### Coating of Highly Loaded PUFA Particle with a Sucrose Layer

The PUFA-coated fumed silica particle produced in Example 24, above, was further coated to produce a barrier layer of solid sucrose. The apparatus was as described in Example 1 with the following operational modifications. Nitrogen was employed as the drying gas and had a nozzle temperature of 302° C. The PUFA oil-coated Cab-O-Sil particles from Example 24 were metered into the apparatus at a rate of 215 g/min. A solution of food grade sucrose (65% w/w in water) was metered into the apparatus at a rate of 71 g/min and at a temperature of 22° C. The dry coated particles were collected as described in Example 1. The resultant particle possessed a first, internal layer of PUFA oil and a second, external layer of sucrose that constituted 17.7% of the finished product. Such a solid coating on a PUFA material is useful as a barrier to the undesirable effects of oxidation and it improves the handling characteristics of the PUFA oil-coated particles.

### Example 27

### Production of PUFA oil Particles with Multiple Coating Layers of Sucrose

PUFA oil loaded particles possessing a single sucrose barrier layer of 14.4% as produced in Example 25 were used as the starting materials for preparation of a series of multiply coated particles.

Using the product of Example 25 as the solid feed material in the coating apparatus produced a particle containing two layers of sucrose. This was achieved by metering the product of Example 25 into the apparatus at a rate of 463 g/min. Nitrogen drying gas was at 414 KPa and was 285° C. at the nozzle. A solution of food grade sucrose (84% w/w in water) was metered into the apparatus at a rate of 82 g/min and at a temperature of 95° C. The dry coated particles were collected as described in Example 1. The particle produced by this process consisted of a Cab-O-Sil core, an internal layer of PUFA oil, and a double layer of sucrose constituting 25.5% of the particle.

A PUFA oil particle that possessed three layers of sucrose was produced by using the doubly coated particle, produced above, as the solid feed material for the coating apparatus. The PUFA particle with 30.2% sucrose was metered into the apparatus at a rate of 634 g/min. Nitrogen drying gas was at 414 KPa and was 277° C. at the nozzle. A solution of food grade sucrose (84% w/w in water) was metered into the apparatus at a rate of 96 g/min and at a temperature of 95° C. The dry coated particles were collected as described in Example 1. The particle produced by this process consisted of a Cab-O-Sil core, an internal layer of PUFA oil, and a triple layer of sucrose constituting 33.9% of the particle.

It is believed that PUFA oil particles possessing multiple barrier layers are particularly resistant to oxidative damage. Oxidation of the PUFA oil is measured using the methods available in the art. Such methods include the Active Oxygen Method, method Cd12-57 of the American Oil Chemists' Society (AOCS), and determination of the Oil Stability Index, AOCS method Cd 12b-92.

### Example 28

### Coating of a Sucrose-Coated PUFA Particle with a High Melting Temperature Fat

A PUFA containing particle was produced with multiple layers of sucrose and an additional barrier layer comprised of high melting temperature fat. The particle with three layers of sucrose (33.9% of the particle weight) produced in Example 27 was metered into the apparatus at a rate of 880 g/min. Nitrogen drying gas was at 414 KPa and was 216° C. at the nozzle. High melting temperature fat (Dritex, ACH Food Companies, Cordova, Tenn.) was metered at 122 g/min. to the center tube at 80° C. using the peristaltic metering pump. The dry coated particles were collected as described in Example 1. The particle produced by this process consisted of a Cab-O-Sil core, an internal layer of PUFA oil, an internal triple layering of sucrose, and an additional barrier layer of high melting fat that constituted 12.2% of the particle.

### Example 29

### Coating of a Sucrose-Coated PUFA Particle with Zein to Provide a Barrier to Moisture

A PUFA containing particle was produced with multiple layers of sucrose and a moisture barrier comprised of dried zein. The particle with three layers of sucrose (33.9% of the particle weight) produced in Example 27 was metered into the apparatus at a rate of 936 g/min. Nitrogen drying gas was at 414 KPa and was 216° C. at the nozzle. A solution of zein (20% w/w in 90% EtOH/10% water, F4000 corn zein, Freeman Industries, Tuckahoe, N.Y.) was metered at 67 g/min. to the center tube at 22° C. using the peristaltic metering pump. The dry coated particles were collected as described in Example 1. The particle produced by this process consisted of a Cab-O-Sil core, an internal layer of PUFA oil, an internal triple layering of sucrose, and an external layer of zein that constituted 1.4% of the particle.

### Example 30

### Coating of a Sucrose-Coated PUFA Particle with Multiple Layers of Zein to Provide a Barrier to Moisture

A PUFA containing particle was produced with multiple layers of sucrose and multiple layers of dried zein. The produced in Example 29, above, was metered into the apparatus at a rate of 939 g/min. Nitrogen drying gas was at 414 KPa and was 293° C. at the nozzle. A solution of zein (20% w/w in 90% EtOH/10% water, F4000 corn zein, Freeman Industries, Tuckahoe, N.Y.) was metered at 192 g/min. to the center tube at 22° C. using the peristaltic metering pump. The dry coated particles were collected as described in Example 1. The particle produced by this process consisted of a Cab-O-Sil core, an internal layer of PUFA oil, an internal triple layering of sucrose, a first layer of zein, and a second, external layer of zein that constituted 5.3% of the particle.

### Examples 31-35

### Loading of a Liquid Drug onto a Solid Carrier

A liquid over the counter cough remedy (Vicks44®, containing one single dilute active pharmaceutical ingredient: dextromethorphan HBr (30 mg dose of active pharmaceutical ingredient in 15 ml), Procter & Gamble, Inc., Cincinnatti, Ohio) was loaded onto solid carrier particles to produce a series of dry flowable drug products in which the drug was present as a coated liquid. The apparatus as shown in FIG. 1 had a mixing chamber 25.4 mm in diameter and 254 mm in length with a nozzle throat of 9.4 mm and a central liquid feed tube of 3.9 mm I.D. The apparatus had a dual screw metering feeder (K-Tron K2VT35 dual 1.5 inch) for metering the solid particles. A peristaltic pump was fit with Masterflex® #25 tubing for metering the liquid. The coating liquid was at ambient temperature and was metered at the feeding rates specified in Table 4 to the center tube using the peristaltic metering pump. Nitrogen gas was supplied to the nozzle at 483 KPa, and was at 25° C. at the nozzle. The nitrogen gas was used to atomize the liquid drug, producing a negative pressure in the mixing zone to induce the addition of the silica particles. Carrier particles used as the solid feed material in the process were silica particles (Syloid®244, approximately 300 m₂ surface area per gram, WR Grace and Co., Lexington, Ky.) fumed silica particles (Cab-O-Sil® EH-5, approximately 600 m₂ surface area per gram, Cabot Corporation, Boston, Mass.). The final composition of the product of each example is given as the weight percentage that is liquid drug.

TABLE 4

| | Drying | Gas | | Solid | Liquid | Liquid | Coating |
|---|---|---|---|---|---|---|---|
| | gas, | nozzle | Solid | feed | feed | feed | on |
| | pressure | temp. | feed | rate | rate | temp. | finished |
| Example | (Kpa) | (° C.) | material | (g/min) | (g/min) | (° C.) | particle (%) |
| 31 | N₂, 483 | 25 | Syloid | 537 | 59 | 25 | 9.9 |
| | | | 244 | | | | |
| 32 | N₂, 483 | 25 | Syloid | 521 | 215 | 25 | 29.2 |
| | | | 244 | | | | |
| 33 | N₂, 483 | 25 | Cab-O-Sil | 507 | 62.8 | 25 | 11 |
| 34 | N₂, 483 | 25 | Cab-O-Sil | 498 | 214.2 | 25 | 30.1 |
| 35 | N₂, 483 | 25 | Cab-O-Sil | 513.6 | 932 | 25 | 64.5 |

The results of these Examples demonstrate that a liquid drug can be loaded onto solid carrier particles, at levels up to 65% of the final material. The resulting particles behave as if they are dry powders and offer a number of advantages for compaction/compression, storage and handling, and for the incorporation of liquid phase drug-containing particles into other dosage forms.

### Examples 36-37

### Loading of an Oil onto a Solid Carrier

Soy oil was loaded onto solid carrier particles to produce a series of dry flowable oil products in which the oil was present as a coated liquid. The apparatus as shown in FIG. 1 had a mixing chamber 25.4 mm in diameter and 254 mm in length with a nozzle throat of 9.4 mm and a central liquid feed tube of 3.9 mm I.D. The apparatus has single screw metering feeder (AccuRate, 2.25 inch) or a dual screw metering feeder (K-Tron K2VT35 dual 1.5 inch) for metering the solid particles. A peristaltic pump was fit with Nalgene 50 or Masterflex® #25 tubing for metering the liquid. The coating liquid was at ambient temperature and was metered at the feeding rates specified in Table 5 to the center tube using the peristaltic metering pump. Nitrogen gas was supplied to the nozzle at the pressure specified in Table 5. The nitrogen gas was used to atomize the oil, producing a negative pressure in the mixing zone to induce the addition of the silica particles. Carrier particles used as the solid feed material in the process were silica particles (Syloid®244, approximately 300 m₂ surface area per gram, WR Grace and Co., Lexington, Ky.). The final composition of the product of each example is given as the percentage that is oil.

TABLE 4

| | Drying | Gas | | Solid | Liquid | Liquid | Coating |
|---|---|---|---|---|---|---|---|
| | gas, | nozzle | Solid | feed | feed | feed | on |
| | pressure | temp. | feed | rate | rate | temp. | finished |
| Example | (Kpa) | (° C.) | material | (g/min) | (g/min) | (° C.) | particle (%) |
| 36 | N₂, 552 | 25 | Syloid | 636 | 94 | 25 | 12.9 |
| | | | 244 | | | | |
| 37 | N₂, 414 | 25 | Syloid | 562 | 278 | 25 | 33.1 |
| | | | 244 | | | | |

The results of these Examples demonstrate that an edible oil can be loaded onto solid carrier particles, at levels up to 30% of the final material. The resulting particles behave as if they are dry powders and offer a number of advantages for compaction/compression, storage and handling, and for the incorporation of oil-containing particles into other dosage forms.

### Example 38

### Coating of an Oil-Loaded Particle with a Gelatin Layer

The oil-coated silica particle produced in Example 36, above, was further coated to produce a barrier layer of solid gelatin. The apparatus was as described in Example 36 with the following operational modifications. Nitrogen was employed as the drying gas and had a nozzle temperature of 241° C. The oil-coated silica particles from Example 36 was metered into the apparatus at a rate of 232 g/min. A solution of food grade gelatin (25% w/w in water) was metered into the apparatus at a rate of 41 g/min and at a temperature of 25° C. The dry encapsulated particles were collected as described in Example 1. The resultant particle possessed a first, internal layer of soy oil and a second, external layer of gelatin that constituted 4.2% of the finished product. Such a solid coating on a oil material is useful as a barrier to the undesirable effects of oxidation and it improves the handling characteristics of the oil-coated particles.

### Example 39

### Coating of an Oil-Loaded Particle with a Casein/Silica Layer

The oil-coated silica particle produced in Example 37, above, was further coated to produce a barrier layer of solid casein/silica. The apparatus was as described in Example 37 with the following operational modifications. Nitrogen was employed as the drying gas and had a nozzle temperature of 230° C. The oil-coated silica particles from Example 37 were metered into the apparatus at a rate of 291 g/min. A suspension of 5% silica (Cab-o-Sil) in a casein solution (10% w/w in water) was metered into the apparatus at a rate of 106 g/min and at a temperature of 25° C. The dry encapsulated particles were collected as described in Example 1. The resultant particle possessed a first, internal layer of soy oil and a second, external layer of casein with Cab-o-Sil silica particles that constituted 5.2% of the finished product. Such a solid coating on an oil material is useful as a barrier to the undesirable effects of oxidation and it improves the handling characteristics of the oil-coated particles.

What is claimed is:

1. A process for coating a polyunsaturated fatty acid (PUFA)-containing carrier particle or a PUFA matrix particle, the process comprising the steps of:
(a) metering a liquid coating material through a flow restrictor;
(b) injecting a gas stream through the flow restrictor concurrently with step (a) to (i) atomize the liquid coating material and (ii) create turbulent flow of the gas stream and the atomized liquid coating material, wherein the gas stream is optionally heated; and
(c) adding a PUFA-containing carrier particle or a PUFA matrix particle to the turbulent flow region concurrently with steps (a) and (b), wherein the PUFA-containing carrier particle or the PUFA matrix particle mixes with the atomized liquid coating material to provide an coated PUFA-containing carrier particle or a PUFA matrix particle.

2. A process for coating a liquid pharmaceutical-containing carrier particle or a liquid pharmaceutical matrix particle, the process comprising the steps of:
(a) metering a liquid coating material through a flow restrictor;
(b) injecting a gas stream through the flow restrictor concurrently with step (a) to (i) atomize the liquid coating material and (ii) create turbulent flow of the gas stream and the atomized liquid coating material, wherein the gas stream is optionally heated; and
(c) adding a liquid pharmaceutical-containing carrier particle or a liquid pharmaceutical matrix particle to the turbulent flow region concurrently with steps (a) and (b), wherein the liquid pharmaceutical-containing carrier particle or the liquid pharmaceutical matrix particle mixes with the atomized liquid coating material to provide an coated pharmaceutical-containing carrier particle or a pharmaceutical matrix particle.

3. The process of claim 1 or 2 wherein the carrier particle is selected from the group consisting of a protein, fumed silica, titanium dioxide and calcium carbonate, a carbohydrate, a food particle, minerals, salts, antioxidants, solid pharmaceutical particles and lipids.

4. The process of claim 2 wherein the pharmaceutical is selected from the group consisting of nutraceuticals, vitamins, supplements, minerals, enzymes, probiotics, bronchodilators, anabolic steroids, analeptics, analgesics, proteins, peptides, antibodies, vaccines, anesthetics, antacids, antihelmintics, anti-arrthymics, antibiotics, anticoagulants, anticolonergics, anticonvulsants, antidepressants, antidiabetics, antidiarrheals, anti-emetics, anti-epileptics, antihistamines, antihormones, antihypertensives, anti-inflammatories, antimuscarinics, antimycotics, antineoplastics, anti-obesity drugs, antiprotozoals, antipsychotics, antispasmotics, anti-thrombics, antithyroid drugs, antitussives, antivirals, anxiolytics, astringents, beta-adrenergic receptor blocking drugs, bile acids, bronchospasmolytic drugs, calcium channel blockers, cardiac glycosides, contraceptives, corticosteriods, diagnostics, digestives, diuretics, dopaminergics, electrolytes, emetics, haemostatic drugs, hormones, hormone replacement therapy drugs, hypnotics, hypoglycemic drugs, immunosuppressants, impotence drugs, laxatives, lipid regulators, muscle relaxants, pain relievers, parasympathicolytics, parasympathicomimetics, prostagladins, psychostimulants, sedatives, sex steroids, spasmolytics, sulfonamides, sympathicolytics, sympathicomimetics, sympathomimetics, thyreomimetics, thyreostatic drugs, vasodialators, and xanthines.

5. The process of claim 1 or 2 wherein the liquid coating material is selected from the group consisting of a natural food color, a synthetic food color, a sugar, a cellulose, a biodegradable polymer, a biodegradable oligomer, an emulsifying wax, a shellac, a flavoring agent, a moisture barrier, a taste-masking agent, an odor-masking agent, hydrophobicity or hydrophilicity agents, a shelf-life extending agent, a lipid, a protein, or a mineral, ethyl cellulose, methyl cellulose, hydroxypropylcellulose, polyvinylpyrolidone, polyethylene, Aquateric, Eudragit™ (including any commercial grade or formulations), acrylic coatings, Surelease™, bubble gum flavor, cherry flavor, grape flavor, sodium lauryl sulfate, sodium docusate, poly lactic acid, poly lactide glycolic acid, cellulose acetate pthalate, diluents, lactose, microcrystalline cellose, mannitol, dicalcium phosphate, starch, dextrates, sucrose, disintegrants, croscarmellose sodium, sodium starch glycolate, carbohydrate; and as binders: hydroxypropyl cellulose, hydroxypyroylmethylcellulose, povidone, methyl cellulose, glidants/lubricants, silicon dioxide, stearic acid, a hydrocolloid, a monosaccharide, a disaccharide, an oligosaccharide, a polysaccharide, a surface modifying agent, a sugar alcohol, a poly-ol, a flow aid, an interparticle force control agent, magnesium stearate, talc, sodium stearyl fumarate, surfactants, sodium lauryl sulfate, Tween 80, Poloxamer®, hydroxypropyl cellulose, hydroxypropylmethylcellulose, titanium dioxide, colors, polyethylene glycols, triethyl citrate, triacetin, dibutyl sebacate and polymethacrylates.

6. The process of claim I wherein the polyunsaturated fatty acid is selected from the group consisting of γ-linolenic acid (GLA), dihomo-γ-linolenic acid, arachidonic acid (ARA), docosahexaenoic acid (DHA) and/or eicosapentaenoic acid (EPA).

7. The process of claim 1 or 2 further comprising repeating steps (a)-(c) at least once wherein the liquid coating material is the same or different or repeating steps (a)-(c) at least once using a liquid coating material which can be the same or different from the liquid coating material.

8. A coated PUFA-containing carrier particle or coated PUFA matrix particle made by the process of any of claims 1,3 5,6 or 7.

9. A coated liquid pharmaceutical-containing carrier particle or coated liquid pharmaceutical matrix particle made by the process of any of claims 2, 3, 4, 5 or 7.

10. A food comprising a coated carrier particle or coated matrix particle made by the process of claims 1 or 2.

11. A nutritional supplement comprising a coated carrier particle or coated matrix particle made by the process of claim 1 or 2.

12. A beverage comprising a coated carrier particle or coated matrix particle made by the process of claims 1 or 2.

13. Infant formula comprising a coated carrier particle or coated matrix particle made by the process of claims 1 or 2.

14. A pet food comprising a coated carrier particle or coated matrix particle made by the process of claims 1 or 2.

15. Animal feed comprising a coated carrier particle or coated matrix particle made by the process of claims 1 or 2.

16. Use of a coated carrier particle or coated matrix particle made by the process of claims 1 or 2 in producing a product selected from the group consisting of a food, nutritional supplement beverage, pharmaceutical formulation, infant formula, pet food or animal feed.

17. Use of a coated carrier particle or coated matrix particle made by the process of claim 2 to administer a pharmaceutical to a mammal by an oral, inhalable, transdermal, parenteral, buccal, nasal, vaginal, rectal, sublingual, ocular, periodontal, implantation, or topical delivery route.
